(19) United States
(12) Patent Application Publication
(10) Pub. No.: US 2023/0397862 A1
(43) Pub. Date: Dec. 14, 2023

(54) Kit For Drawing And Collecting Blood

(71) Applicant: Hennesys Disla, Buffalo, NY (US)

(72) Inventor: Hennesys Disla, Buffalo, NY (US)

(21) Appl. No.: 18/208,673

(22) Filed: Jun. 12, 2023

Related U.S. Application Data
(60) Provisional application No. 63/351,253, filed on Jun. 10, 2022.

(51) Int. Cl. A61B 5/153; A61B 5/15

(52) U.S. Cl. CPC A61B 5/153; A61B 5/15003; A61B 5/150366

(57) ABSTRACT

The present invention provides a portable kit for blood collection. The kit contains the essential components for drawing blood from a person using a needle and blood collection tube. These components preferably include multiple blood collection tubes, needles, needle holders, gauze pads, bandages, and alcohol-prep pads. The kit containing the components is lightweight, compact, and easily stackable. The kit is portable so a phlebotomist, nurse, or other health care practitioner can easily carry the kit.

### CROSS-REFERENCE TO RELATED APPLICATIONS

The application claims the benefit of U.S. Provisional Patent Application No. 63/351,253 filed Jun. 10, 2022, the entire disclosure of which is hereby incorporated by reference.

### BACKGROUND OF THE INVENTION

### Field of the Invention

The present invention generally relates to a portable kit for blood collection. More particularly, the kit is portable so a phlebotomist, nurse, or other health care practitioner can easily carry the kit. The kit of this invention can be used to draw blood quickly and easily from a patient.

### Brief Review of the Related Art

In general, phlebotomy refers to the drawing of blood from a blood vessel and is also commonly referred to as blood draw or venipuncture. Normally, blood is taken from a vein in the arm. The present invention provides several advantages over conventional blood draw kits. For example, many traditional blood-draw kits do not contain all of the supplies needed to completely perform blood draw procedures. Thus, the health care practitioner must purchase some pieces separately. Also, many conventional blood-draw kits on the market, once opened, must be thrown out even if all of the pieces inside of the kit are not used. Once opened, these kits are considered tainted. The kits cannot be re-sealed.

In contrast to many blood draw kits on the market, the phlebotomy kits of the present invention contain all of the necessary pieces to complete a blood draw. The blood draw kits of the present invention are low-cost and of high quality. The kits are lightweight, compact, and easily stackable.

Another advantage of the kits of the present invention is that the medical supplies in the kit are vacuum-sealed in translucent bags. Thus, the medical supplies are plainly visible. The medical supplies are purposely organized in the kit so that the health care practitioner can easily locate and use them. The health care practitioner does not have to worry about rustling around the box looking for a piece. The health care practitioner can safely carry the kit to and from a clinic knowing that all of the pieces needed to perform a health draw are safely stored in the kit. Yet another benefit is that the health care practitioner can open and close the kit easily. This reduces the possibility of throwing out medical supplies that they might not use immediately. Once the kit is opened, it can be re-sealed and any unused products inside of the kit can be saved and used at the next blood draw procedure. This helps reduce hospital and clinical waste. The blood draw kits of the present invention provide additional features, benefits and advantages as described further below.

### SUMMARY OF THE INVENTION

The present invention provides a portable kit for collecting blood. The kit comprises: a) a blood collection tube comprising an elongated tube having an open end and a closed end, wherein there is rubber stopper fitted over the open end; b) a needle comprising an outer end for inserting into the blood vessel and an inner end for inserting into the blood collection tube through the rubber stopper; c) a translucent holder for the blood collection tube, the holder having a first end for inserting the needle and a second end for inserting the blood collection tube; and a material selected from the group consisting of a gauze pad, bandage, alcohol-prep pad, and tourniquet, and combinations thereof.

A specimen bag can be included in the kit. In one preferred embodiment, the kit further comprises gloves. Non-latex gloves can be used. In another preferred embodiment, the kit contains a face mask. The kit preferably contains multiple needles and blood collection tubes. The needles are preferably vacuum-sealed in translucent bags inside of the kit.

### BRIEF DESCRIPTION OF THE DRAWINGS

The novel features that are characteristic of the present invention are set forth in the appended claims. However, the preferred embodiments of the invention, together with further objects and attendant advantages, are best understood by reference to the following detailed description in connection with the accompanying drawings in which:

FIG. 1 is a top view of one embodiment of a kit of the present invention;

FIG. 2 is a top view of a second embodiment of a kit of the present invention;

FIG. 3 is a top view of a third embodiment of a kit of the present invention;

FIG. 4 is a top view of a fourth embodiment of a kit of the present invention; and

FIG. 5 is a top view of a fifth embodiment of a kit of the present invention.

### DETAILED DESCRIPTION OF THE INVENTION

The present invention generally relates to a portable kit for blood collection. The kit contains the essential components for drawing blood from a person using a needle and blood collection. The kit containing the components is lightweight, compact, and easily stackable. The kit is portable so a phlebotomist, nurse, or other health care practitioner can easily carry the kit. Students can use the kit for practicing blood draws. The kit of this invention can be used to draw blood quickly and easily from a patient. The drawn blood is collected in blood collection tubes. These components are described as follows.

Blood Collection Tubes: The kit of the present invention contains blood collection tubes (10) that are used to collect blood from the patient. The tubes are filled with a predetermined volume of blood using a needle and vacuum process as described further below. The elongated blood collection tubes have an open end and a closed end, wherein there is a rubber stopper fitted over the open end. The rubber stoppers are color coded according to the additive that the tube contains.

Blood collection tubes must be drawn in a specific order to avoid cross-contamination of additives between the tubes. The recommended order of draw is as follows:
1.) Blood culture bottle or tube (yellow or yellow-black top)
2.) Coagulation tube (light blue top). If just a routine coagulation assay is the only test ordered, then a single light blue top tube may be drawn. If there is a concern regarding contamination by tissue fluids or thromboplastins, then one may draw a non-additive tube first, and then the light blue top tube.

3.) Non-additive tube (red top)
4.) Additive tubes following this order: i) SST (red-gray or gold top). Contains a gel separator and clot activator; ii) sodium heparin (dark green top); iii) PST (light green top). Contains lithium heparin anticoagulant and a gel separator; iv) EDTA (lavender top); v) ACDA or ACDB (pale yellow top). Contains acid citrate dextrose; and vi) Oxalate/fluoride (light gray top).

Any suitable blood collection tube can be used in accordance with the present invention. For example, Vacutainer® brand sterile hematology tubes with the Hemogard™ closure can be used. These closures help to protect lab personnel from contact with blood on the stopper or around the outer rim of the tube, as well as from blood splattering upon opening the tube. The rubber stopper is recessed inside the plastic shield so any drops left by a blood collection needle remain isolated from potential contact. The blood collection tubes can have various sizes. In one example, the blood collection tube has a diameter of about 13 mm and length of about 75 mm, Glass and plastic blood collection tubes can be used.

Needles: Typically, the needles have opposing first and second ends (tips), wherein the first (outer) end is inserted into the vein. While the second (inner) end is inserted into the blood collection tube. Any suitable needle can be used in accordance with this invention. For example, commercially-available needles from Becton Dickinson (BD) can be used in accordance with this invention. The needles should be able to protect healthcare workers from needlestick injuries and exposure to bloodborne pathogens. The needles are described in further detail below.

Holders: The needle may be screwed into a translucent plastic holder. Inserting the blood collection tube into the holder causes the inner tip of the needle to puncture the rubber cap of the collection tube. Then, the vacuum in the tube pulls the blood through the needle and into the tube. The amount of air evacuated from the tube predetermines how much blood will fill the tube before blood stops flowing. Once the collection tube is filled with blood, it can be removed and placed in a specimen bag or other secure contained. Such specimen bags are discussed further below. If necessary, the phlebotomist can insert another collection tube and draw another sample of blood in the same way. In some cases, the needle can have a thin rubber coating that helps prevent blood from leaking out if tubes are changed. The gauge number of the needle indicates the bore size: the larger the gauge number, the smaller the needle bore. That is, as the needle gauge increases, the needle width becomes narrower. Needles having gauges of 21, 22, and 23 are the most common. Needles having a 23 gauge are also known as butterfly needles and are used on patients having very small veins.

Gauze Pads: After the blood has been drawn, a gauze pad should be placed over the puncture site on the skin; and the needle should be withdrawn. Any suitable gauze pad can be used in accordance with this invention. For example, woven gauze pads can be used. These gauze pads are typically light, thin, and made of loosely woven pieces of fabric. Nonwoven gauze pads also can be used. The nonwoven gauze contains fibers pressed together to resemble a weave. The loose, open weave of these pads allows the blood to be absorbed into the fibers. These pads have high absorbency. For example, gauze pads available from Dukal Corp. (Ronokoma, NY 11779) can be used. Common sizes include 2 in ×2 in; 3 in ×3 in; and 4 in ×4 inch gauze. After the gauze pad is placed in position, slight pressure is applied to the pad. When the bleeding stops, a fresh bandage can be applied over the puncture site.

Bandages: An adhesive bandage can be applied over the wound. Any suitable bandages can be used. For example, Band-Aids®, which are commercially available from Johnson & Johnson can be used.

Alcohol-Prep Pads: The puncture site on the arm or other area of the patient is cleaned with an alcohol-prep pad before making the vein puncture with the needle. The alcohol pad works as an antiseptic cleaner, Any suitable alcohol pad can be used in accordance with this invention. In one embodiment, non-woven, 2-ply, medium-sized alcohol pads that are saturated with isopropyl alcohol (70%) can be used. These alcohol pads are available in packs, wherein there are 200 pads per pack.

Tourniquet: The tourniquet is normally applied about 3 to about 4 inches above the collection site. The elastic tourniquet produces a temporary increase in blood pressure. Any suitable tourniquet can be used in accordance with this invention. For example, Latex-Free Tourniquets from MedLine can be used. These tourniquets are individually rolled and banded. This makes it easy to grab just one tourniquet. In addition, these tourniquets are safe for use on patients with latex sensitivities allowing for use on a wider range of patients.

Face mask: The kits also may contain face masks for the health care practitioner to wear. Preferably, the face mask contains three breathable layers. The face mask is used to prevent exposure to blood borne pathogens.

Gloves: Latex or non-latex gloves also can be included in the kit and these gloves can be used to prevent the phlebotomist or other health care practitioner from contacting the blood.

Specimen bag: The blood, which is in the collection tubes, can be transferred using recloesable Biohazard Specimen Transport Bags. Anu suitable blood transport specimen bag can be used. For example, bags having 3-Ply 6 in ×9 in ×2 Mils with an outer pouch for paperwork can be used. Reclosable Zip-Lock™ biohazard specimen bags also can pre-printed with labels or other markings. These bags are constructed with reinforced side-welded zippers to lock the bags in a smooth motion. The specimen bags should Meet FDA/USDA specification. In some embodiments, the bags are made of pure Low-Density Polyethylene (LDPE) resin. These bags can be store at temperatures less than 120° F. The bags should not be stored in direct sunlight.

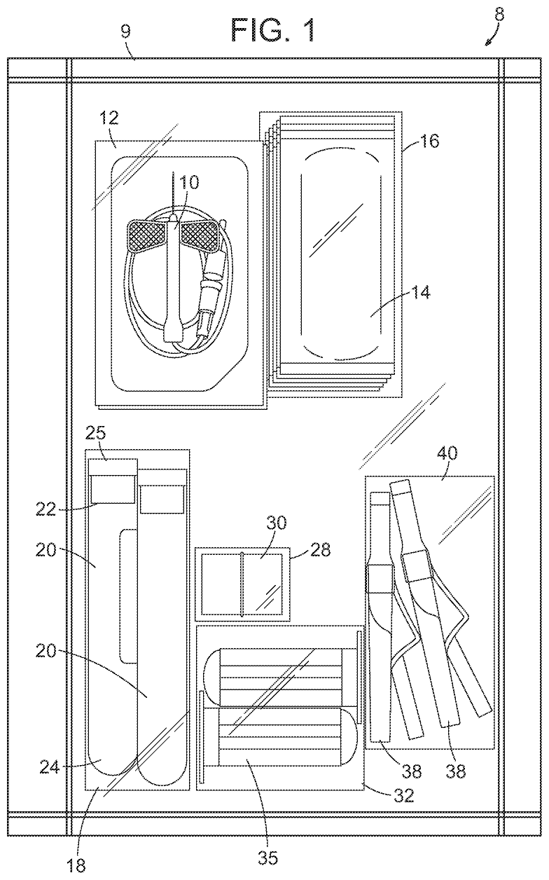

Referring to FIG. 1, one embodiment of a kit (8) of the present invention (which can be referred to as “Starter Kit C”) is shown. The medical supplies are specifically positioned in the kit (8) so that a health care practitioner can easily locate and use them. The medical supplies can be enclosed with separate vacuum-sealed bags or pockets inside of the kit (8). In particular, the kit (8) of FIG. 1 has an interior compartment containing two butterfly needles (10) enclosed within a first sealed pocket (12). The interior compartment may be defined by sealed packaging (9) that encloses the sealed pocket(s) and components. The sealed packaging (9) overlies the sealed pocket(s) and components of the kit. There are five bandages (14) overlying five alcohol-prep pads (not shown) in a second sealed pocket (16), which is adjacent to the first sealed pocket (12). In a third sealed pocket (18), which is located below the first and second sealed pockets, there are two blood collection tubes (20). The elongated blood collection tubes (20) have an open end (22) and a closed end (24), wherein there is a rubber stopper (25) fitted over the open end. As discussed above, the rubber stoppers are color coded.

A fourth sealed pocket (28) contains a tourniquet (30), while a fifth sealed pocket (32) contains two translucent needle chambers (holders) generally indicated at (35). Finally, there are two straight needles (38) located in a sixth sealed pocket (40). This distinct organization of the medical supplies in the kit is a key advantageous feature of the kits of the present invention. With the medical supplies being distinctly and separately positioned, the health care practitioner knows where each component is located. The practitioner does not have to spend time and energy rustling around the kit in search of a particular blood drawing tool.

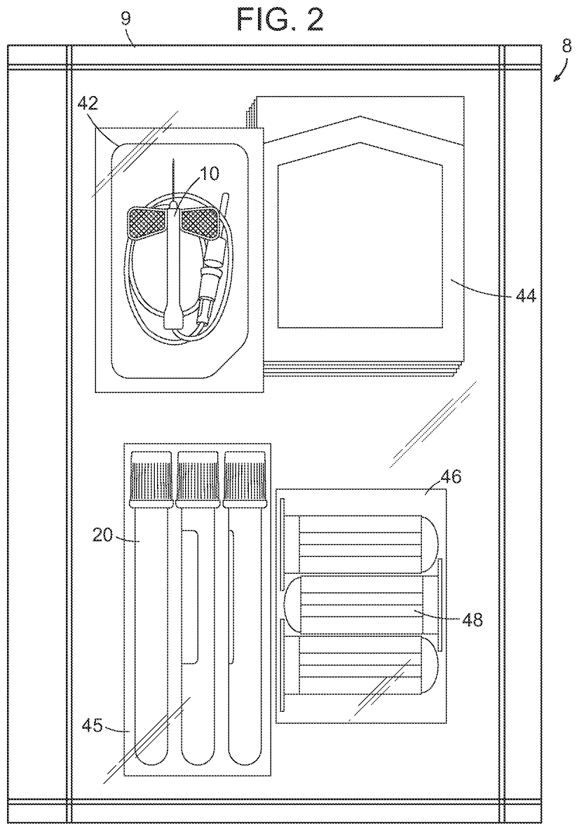

Turning to FIG. 2, a second embodiment of a kit (8) is shown. This kit (8) contains a first sealed pocket (42) containing one butterfly needle (10) and five gauze pads (44). There are three blood collection tubes (20) located in a second sealed pocket (45), which is located below the first sealed pocket (42). A third sealed pocket (46) contains three needle chambers (holders) (48). The kit comprises sealed packaging (9), that is, the kit is wrapped in translucent plastic packaging material that encloses the sealed pocket(s) and components of the kit. The sealed packaging (9) overlies the sealed pocket(s) and components of the kit. Vacuum-sealed bags can also be used to hold the components of the kit (8).

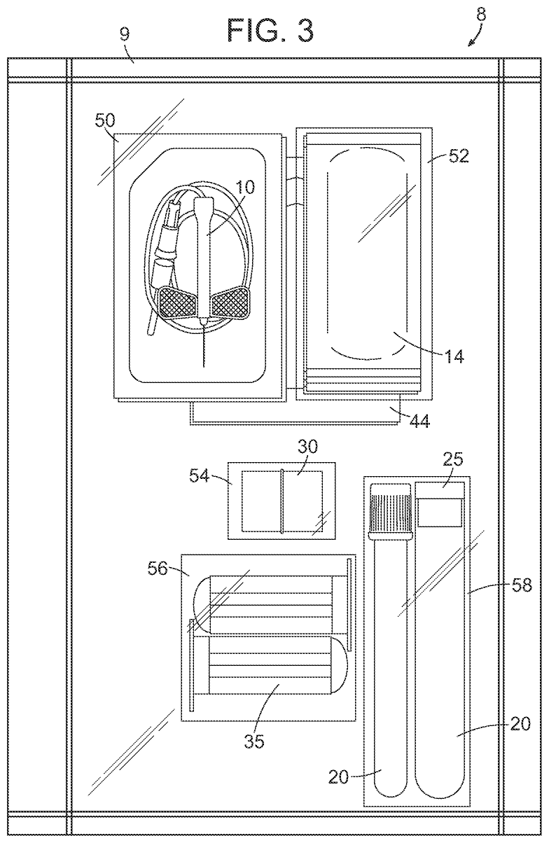

Referring to FIG. 3, a third example of a kit (8) of the present invention (which can be referred to as “Starter Kit B”) is shown. This kit (8) also contains various medical supplies arranged in a sealed package (9). The medical supplies can be enclosed with separate vacuum-sealed bags or pockets inside of the kit (8). For example, the medical supplies may be wrapped and enclosed within separately sealed pockets that are enclosed within the sealed package. In particular, the kit (8) of FIG. 3, contains two butterfly needles (10) arranged in a first sealed pocket (50). A second sealed pocket (52), which is adjacent to the first sealed pocket, contains two bandages (14), two alcohol-prep pads (not shown), and two gauze pads (44). A third sealed pocket (54), which is below the first and second sealed pockets, contains a tourniquet (30); while a fourth sealed pocket (56), which is below the third sealed pocket, contains two translucent needle chambers (holders) (35). There is also a fifth sealed pocket (58) located below the first and second sealed pockets; and this pocket holds two blood collection tubes (20).

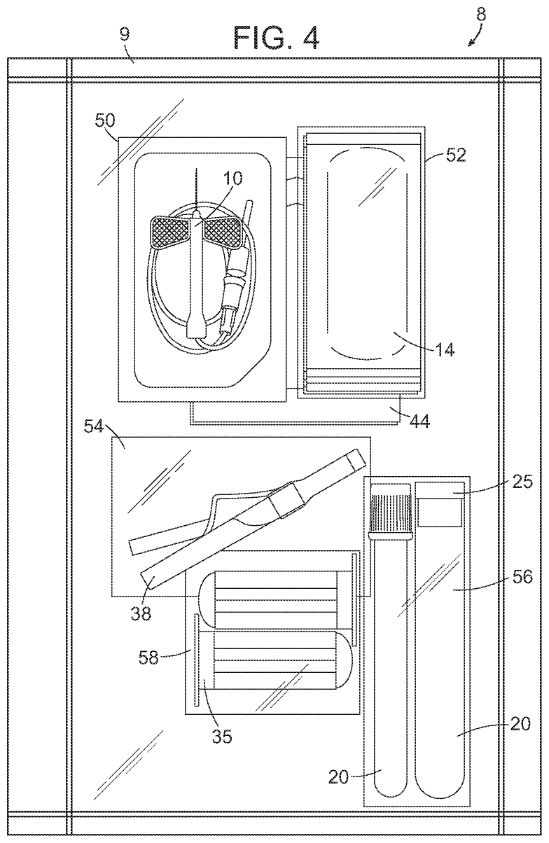

In FIG. 4, a fourth example of a kit (8) of the present invention (which also can be referred to as “Starter Kit B”) is shown. This kit (8) also contains various medical supplies arranged in a sealed package (9). The medical supplies can be enclosed with separate vacuum-sealed bags or pockets inside of the kit (8). For example, the medical supplies may be wrapped and enclosed within separately sealed pockets that are enclosed within the sealed package. In particular, the kit (8) of FIG. 4 contains a first sealed pocket (50) containing one butterfly needle (10). There is an adjacent second sealed pocket (52) containing two bandages (14), two alcohol-prep pads (not shown), and two gauze bandages (44). There are three sealed pockets located below the first and second sealed pockets. The third sealed pocket (54) contains one straight needle (38). In a fourth sealed pocket (56), there are two blood collection tubes (20). In a fifth sealed pocket (58), there are two translucent needle chambers (holders) (35).

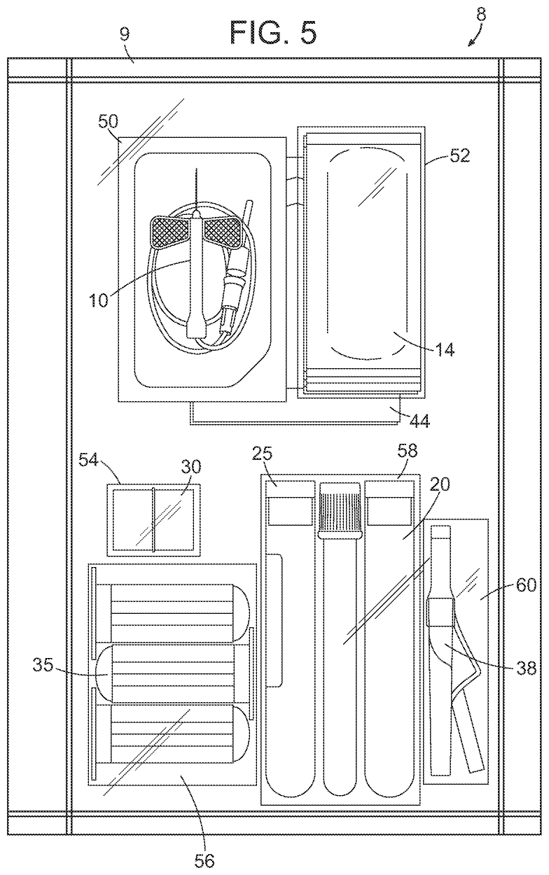

Lastly, referring to FIG. 5, a fourth example of a kit (8) of the present invention (which also can be referred to as “Starter Kit A”) is shown. This kit (8) also contains various medical supplies arranged in a sealed package (9). The medical supplies can be enclosed with separate vacuum-sealed bags or pockets inside of the kit (8). The sealed packaging of the kit encloses separately sealed pockets, and the pockets hold various components of the kit. In particular, the kit in FIG. 5 has a first sealed pocket (50) containing one butterfly needle (10). There is a second sealed pocket (52) adjacent to the first sealed pocket, and this second pocket contains three bandages (14) overlying three alcohol-prep pads (not shown). The sealed pocket (52) also contains three gauze pads (44). A third sealed pocket (54), which is below the first and second sealed pockets, contains a tourniquet (30); while a fourth sealed pocket (56), which is below the third sealed pocket, contains three translucent needle chambers (holders) (35). There is also a fifth sealed pocket (58) located below the first and second sealed pockets; and this pocket holds three blood collection tubes (20). A sixth sealed pocket (60), which is adjacent to the fifth sealed pocket, contains two straight needles (38).

It should be understood the terms, “first”, “second”, “third”, “fourth”, “fifth”, “sixth” “top”, “bottom”, “below”, “upper”, “lower”, “upper”, “downward”, “right”, “left”, “anterior”, “posterior”, and the like are arbitrary terms used to refer to one position of an element based on one perspective and should not be construed as limiting the scope of the invention.

It also should be understood that the blood collection kit, components of the kit, constructions, materials, and the like described and illustrated herein represent only some embodiments of the invention. It is appreciated by those skilled in the art that various changes and additions can be made to the kits, components, constructions, materials, and the like without departing from the spirit and scope of this invention. It is intended that all such embodiments be covered by the appended claims.

We claim:

1. A portable kit for collecting blood, the kit comprising:
a) a blood collection tube comprising an elongated tube having an open end and a closed end, wherein there is rubber stopper fitted over the open end;
b) a needle comprising an outer end for inserting into the blood vessel and an inner end for inserting into the blood collection tube through the rubber stopper;
c) a holder for the blood collection tube, the holder having a first end for inserting the needle and a second end for inserting the blood collection tube;
d) at material selected from the group consisting of a gauze pad, bandage, alcohol-prep pad, and tourniquet, and combinations thereof.

2. The portable kit of claim 1, wherein the kit has an interior compartment, the compartment enclosing the blood collection tube, needle, holder of the blood collection tube, and material selected from the group consisting of a gauze pad, bandage, alcohol-prep pad, and tourniquet, and combinations thereof.

3. The portable kit of claim 1, wherein the holder for the blood collection tube is translucent.

4. The portable kit of claim 1, further comprising a specimen bag.

5. The portable kit of claim 1, further comprising gloves.

6. The portable kit of claim 1, further comprising a face mask.

7. The portable kit of claim 1, wherein the kit comprises multiple needles, blood collection tubes, and holders for the blood collection tubes.

8. The portable kit of claim 7, wherein the needles, blood collection tubes, and holders for the blood collection tubes are vacuum-sealed within separate translucent bags, and the translucent bags are enclosed within the kit.

9. The portable kit of claim 1, wherein the kit comprises multiple gauze pads; bandages; and alcohol-prep pads.

10. The portable kit of claim 9, wherein the gauze pads, bandages, and alcohol-prep pads are enclosed within vacuum-sealed translucent bags inside of the kit.

11. The portable kit of claim 1, wherein the kit comprises sealed packaging, the sealed packaging enclosing components of the kit.

12. The portable kit of claim 12, wherein the components of the kit are selected from the group consisting of blood collection tubes, needles, translucent holders for the blood collection tubes, gauze pads, bandages, alcohol-prep pads, and tourniquets, and combinations thereof.

13. The portable kit of claim 1, wherein the kit comprises sealed packaging, the sealed packaging enclosing separately sealed multiple pockets, wherein each pocket holds at least one component of the kit.

14. The portable kit of claim 13, wherein the components of the kit are selected from the group consisting of blood collection tubes, needles, holders for the blood collection tubes, gauze pads, bandages, alcohol-prep pads, and tourniquets, and combinations thereof.
